(19) United States
(12) Patent Application Publication
(10) Pub. No.: US 2021/0172962 A1
(43) Pub. Date: Jun. 10, 2021

(54) CES-2 (CARBOXYLESTERASE-2) FOR THE ASSESSMENT OF AFIB RELATED STROKE

(71) Applicants: Roche Diagnostics Operations, Inc., Indianapolis, IN (US); UNIVERSITEIT MAASTRICHT, LK Maastricht (NL); AKADEMISCH ZIEKENHUIS MAASTRICHT, HX Maastricht (NL)

(72) Inventors: Manuel Dietrich, Mannheim (DE); Peter Kastner, Penzberg (DE); Vinzent Rolny, Penzberg (DE); Ulrich Schotten, LK-Maastricht (NL); Ursula-Henrike Wienhues-Thelen, Penzberg (DE); Andre Ziegler, Rotkreuz (CH)

(21) Appl. No.: 17/172,761

(22) Filed: Feb. 10, 2021

Related U.S. Application Data
(63) Continuation of application No. PCT/EP2019/071482, filed on Aug. 9, 2019.

(30) Foreign Application Priority Data
Aug. 10, 2018 (EP) 18188437.0

(51) Int. Cl. G01N 33/68

(52) U.S. Cl. CPC G01N 33/6893; G01N 2800/50; G01N 2496/00; G01N 2333/475; G01N 2333/918; G01N 2333/58; G01N 2333/4745; G01N 2800/52

(57) ABSTRACT

The present invention relates to a method for assessing the risk of stroke in a subject, said method comprising the steps of determining the amount of CES-2 in a sample from the subject, and comparing the amount of CES-2 to a reference amount, whereby the risk of stroke is to be assessed. Moreover, the present invention relates to a method for assessing the efficacy of an anticoagulation therapy and a method for identifying a subject being eligible to the administration of at least one anticoagulation medicament or being eligible for increasing the dosage of at least one anticoagulation medicament.

This application is a continuation application and claims priority to PCT/EP2019/071482, filed Aug. 9, 2019, which claims priority to EP 18188437.0, filed Aug. 10, 2018, both of which are incorporated herein in their entireties.

The present invention relates to a method for assessing the risk of stroke in a subject, said method comprising the steps of determining the amount of CES-2 in a sample from the subject, and comparing the amount of CES-2 to a reference amount, whereby the risk of stroke is to be assessed. Moreover, the present invention relates to a method for diagnosing heart failure and/or at least one structural or functional abnormality of the heart associated with heart failure.

### BACKGROUND SECTION

Stroke ranks after ischemic heart disease second as a cause of lost disability life years in high income countries and as a cause of death worldwide. In order to reduce the risk of stroke, anticoagulation therapy appears the most appropriate therapy.

Atrial fibrillation (AF) is an important risk factor for stroke (Hart et al., Ann Intern Med 2007; 146(12): 857-67; Go A S et al. JAMA 2001; 285(18): 2370-5). Atrial fibrillation is characterized by irregular heart beating and often starts with brief periods of abnormal beating that can increase over time and may become a permanent condition. An estimated 2.7-6 1 million people in the United States have atrial fibrillation and approximately 33 million people globally (Chugh S. S. et al., Circulation 2014; 129:837-47).

It is important to assess which patients with AF have the highest risk of atrial fibrillation and thus may benefit from an intensified anticoagulation therapy to reduce the risk of stroke (Hijazi et al., European Heart Journal doi:10.1093/eurheartj/ehw054. 2016).

The CHADS2, the CHA2DS2-VASc score, and the ABC score are clinical prediction rules for estimating the risk of stroke in patients with atrial fibrillation. The scores are used to assess whether or not treatment is required with anticoagulation therapy. The ABC-stroke score includes age, biomarkers (N-terminal fragment B-type natriuretic peptide and high-sensitivity cardiac troponin), and clinical history (prior stroke), see Oldgren et al., Circulation. 2016; 134:1697-1707).

Mammalian carboxylesterases (CESs) comprise a multigene family. They are members of an α, β-hydrolase-fold family and are found in various mammals and are primarily microsomal enzymes (Hosokawa et al. 2007; Satoh and Hosokawa 2006). CESs generally mediate a detoxification process as the resulting metabolites are more hydrophilic and hence more readily excreted. The enzymes encoded by these genes are responsible for the hydrolysis of ester- and amide-bond-containing drugs such as cocaine and heroin. They also hydrolyze long-chain fatty acid esters and thioesters.

CESs can be classified into five major groups, CES1-5, according to the homology of the amino acid sequence, and the majority of CESs that have been identified belong to the CES1 or CES-2 family Carboxylesterase hydrolysis has been utilized in the development of oral prodrugs. For example, CES1 and 2 were described to play a role in the hydrolysis of pro drug Dabigatran Etexilate DABE into active drug metabolite Dabigatran, an oral anticoagulant (Laizure et al. Drug Metab Dispos 42:201-206, February 2014).

CES-2 is a 60-kDa monomer and also known as Carboxylesterase 2; CES-2; iCE; CE-2; PCE-2; CES-2A1. The CES-2 isozyme recognizes a substrate with a large alcohol group and a small acyl group (Satoh and Hosokawa 2006).

CES-2 is predominantly expressed in the small intestine. Furthermore, it is expressed among others in heart, brain, testis, skeletal muscle, colon, spleen, kidney and liver, but considerably less expressed in fetal tissues (e.g. fetal heart, kidney, spleen, and liver) and cancer cells. The human CES-2 has 12 transcripts (splice variants). Wu et al., (Pharmacogenetics. 2003 July; 13(7):425-35) identified three different promoters, wherein two promoters are tissue specific and a further distal promoter is responsible for low level expression of the gene in many tissues.

However, the involvement of CES-2 in cardiovascular conditions, in particular in atrial fibrillation, heart failure and stroke remains unknown.

The prediction of stroke and the selection of preventive medication are important clinical unmet needs. Up to now, CES-2 has not been used to predict the stroke in patients and assessing the efficacy of an anticoagulation therapy.

There is a need for reliable methods for the prediction of stroke, for assessing the efficacy of an anticoagulation therapy, for identifying a subject being eligible to the administration of at least one anticoagulation medicament or being eligible for increasing the dosage of at least one anticoagulation medicament, for monitoring a subject receiving an anticoagulation therapy and for diagnosing heart failure.

The technical problem underlying the present invention can be seen as the provision of methods for complying with the aforementioned needs. The technical problem is solved by the embodiments characterized in the claims and herein below.

Advantageously, it was found in the context of the studies of the present invention that the determination of the amount of CES-2 and/or the amount of one or more of the biomarkers comprising a natriuretic peptide, ESM-1, ANG-2, IGFBP7 in a sample from a subject allows for stroke prediction.

### BRIEF SUMMARY OF THE PRESENT INVENTION

The present method for predicting the risk of stroke in a subject, comprising the steps of
a) determining the amount of CES-2 and optionally one or more biomarkers comprising of a natriuretic peptide, ESM-1, ANG-2, IGFBP7 in a sample from the subject, and
b) comparing the amount of CES-2 and optionally one or more biomarkers comprising of a natriuretic peptide, ESM-1, ANG-2, IGFBP7, to a reference amount (or to reference amounts), whereby the risk of stroke is to be predicted.

In an embodiment of the method of the present invention, the amount of CES-2 in the sample from the subject is decreased as compared to the reference amount (or to reference amounts).

In an embodiment of the method of the present invention, the method further comprising the step of recommending anticoagulation therapy or of recommending an intensification of anticoagulation therapy if the subject has been identified to be at risk to suffer from stroke.

In an embodiment of the method of the present invention, the subject suffers from atrial fibrillation.

In an embodiment of the method of the present invention, the atrial fibrillation is paroxysmal, persistent or permanent atrial fibrillation.

In an embodiment of the method of the present invention, the subject has a history of stroke or TIA (transient ischemic attack)

In an embodiment of the method of the present invention, the age of the subject is 65 years of age or older. Further, the age of the subject may be 55 years or older.

In an embodiment of the method of the present invention, the subject receives anticoagulation therapy.

In an embodiment of the method of the present invention, stroke is cardioembolic stroke.

In an embodiment of the method of the present invention, the subject is human.

In an embodiment of the method of the present invention, the sample is blood, serum or plasma.

In an embodiment of the method of the present invention, amounts of CES-2 and one or more biomarkers comprising of a natriuretic peptide, ESM-1, ANG-2, IGFBP7 are determined in step a), and wherein the method comprises the further steps of c) calculating a ratio of the amount of one or more biomarkers comprising of the natriuretic peptide, ESM-1, ANG-2, IGFBP7 as determined in step a) to the amount of CES-2 as determined in step a), and comparing said calculated ratio to a reference ratio.

The present invention further concerns a method for predicting the risk of stroke in a subject, comprising the steps of
a) determining the amount of CES-2 and/or the amount of one or more biomarkers comprising of a natriuretic peptide, ESM-1, ANG-2, IGFBP7 in a sample from the subject having a known clinical stroke risk score, and
b) assessing the clinical stroke risk score for said subject, and
c) predicting the risk of stroke based on the results of steps a) and b).

The present invention further relates to a method for improving the prediction accuracy of a clinical stroke risk score for a subject, comprising the steps of
a) determining the amount of CES-2 and/or the amount of one or more biomarkers comprising of a natriuretic peptide, ESM-1, ANG-2, IGFBP7, and
b) combining a value for the amount of CES-2 and/or the amount of one or more biomarkers comprising of a natriuretic peptide, ESM-1, ANG-2, IGFBP7 with the clinical stroke risk score, whereby the prediction accuracy of said clinical stroke risk score is improved.

The present invention further relates to a method for assessing the efficacy of an anticoagulation therapy of a subject, comprising the steps of
a) determining the amount of CES-2 and optionally one or more biomarkers comprising of a natriuretic peptide, ESM-1, ANG-2, IGFBP7 in a sample from the subject, and
b) comparing the amount of CES-2 and optionally one or more biomarkers comprising of a natriuretic peptide, ESM-1, ANG-2, IGFBP7 to a reference amount (or to reference amounts), whereby the risk of stroke is to be assessed.

In an embodiment of the method of the present invention, a decreased amount of CES-2 is significant that anticoagulation therapy is not efficient, and wherein a normal or an increased amount of CES-2 is significant that anticoagulation therapy is effective.

The present invention further concerns a method for identifying a subject being eligible to the administration of at least one anticoagulation medicament or being eligible for increasing the dosage of at least one anticoagulation medicament, comprising
a) determining the amount CES-2 and optionally one or more biomarkers comprising of a natriuretic peptide, ESM-1, ANG-2, IGFBP7 and
b) comparing the amount as determined in step a) with a reference amount, whereby a subject being eligible to the administration of said at least one medicament or to an increased dosage of said at least one medicament is identified.

The present invention further relates to a method for monitoring a subject receiving an anticoagulation therapy, comprising the steps of
a) determining the amount of CES-2 and optionally one or more biomarkers comprising of a natriuretic peptide, ESM-1, ANG-2, IGFBP7 in a sample from the subject, and
b) comparing the amount of CES-2 and optionally of one or more biomarkers comprising of a natriuretic peptide, ESM-1, ANG-2, IGFBP7 to a reference amount (or to reference amounts), whereby the risk of stroke is to be assessed.

The present invention further relates to the use of
i) the biomarker CES-2 and optionally of one or more biomarkers comprising of a natriuretic peptide, ESM-1, ANG-2, IGFBP7
ii) at least one detection agent that specifically binds to CES-2, and optionally at least one detection agent that specifically binds to one or more biomarkers comprising of a natriuretic peptide, ESM-1, ANG-2, IGFBP7 in a sample from a subject for a) assessing the risk of stroke or b) for assessing the efficacy of an anticoagulation therapy or c) monitoring a subject receiving an anticoagulation therapy.

The present invention further relates to the use of
i) the biomarker CES-2 and/or
ii) at least one detection agent that specifically binds to CES-2, in a sample from a subject, in combination with a clinical stroke risk score, for predicting the risk of a subject to suffer from stroke.

The present invention further relates to the use of
i) the biomarker CES-2 and/or
ii) at least one detection agent that specifically binds to CES-2 in a sample from a subject for predicting the efficacy of an anticoagulation therapy of a subject.

The present invention further concerns to a kit comprising an agent which specifically binds to CES-2 and an agent which specifically binds to one or more biomarkers comprising of a natriuretic peptide, ESM-1, ANG-2, IGFBP7.

In an embodiment, the detection agent that specifically binds CES-2 is an antibody or antigen binding fragment thereof that specifically binds CES-2.

### DETAILED DESCRIPTION OF THE PRESENT INVENTION —DEFINITIONS

As set forth above, the present method for predicting the risk of stroke in a subject, comprising the steps of
a) determining the amount of CES-2 and optionally one or more biomarkers comprising of a natriuretic peptide, ESM-1, ANG-2, IGFBP7 in a sample from the subject, and
b) comparing the amount of CES-2 and optionally one or more biomarkers comprising of a natriuretic peptide, ESM-1, ANG-2, IGFBP7, to a reference amount (or to reference amounts), whereby the risk of stroke is to be predicted.

The prediction of stroke shall be based on the results of the comparison step (b).

Accordingly, the method of the present invention preferably comprises the steps of
a) determining the amount of CES-2 and optionally one or more biomarkers comprising of a natriuretic peptide, ESM-1, ANG-2, IGFBP7 in a sample from the subject, and
b) comparing the amount of CES-2 and optionally one or more biomarkers comprising of a natriuretic peptide, ESM-1, ANG-2, IGFBP7, to a reference amount (or to reference amounts), whereby the risk of stroke is to be predicted, and
c) predicting the risk of stroke of a subject, preferably based on the results of the comparison step (b).

The method as referred to in accordance with the present invention includes a method which essentially consists of the aforementioned steps or a method which includes further steps. Moreover, the method of the present invention, preferably, is an ex vivo and more preferably an in vitro method. Moreover, it may comprise steps in addition to those explicitly mentioned above. For example, further steps may relate to the determination of further markers and/or to sample pre-treatments or evaluation of the results obtained by the method. The method may be carried out manually or assisted by automation. Preferably, step (a), (b) and/or (c) may in total or in part be assisted by automation, e.g., by a suitable robotic and sensory equipment for the determination in step (a) or a computer-implemented calculation in step (b).

As will be understood by those skilled in the art, the prediction made in connection with present invention is usually not intended to be correct for 100% of the subjects to be tested. The term, preferably, requires that a correct assessment (such as the diagnosis, differentia-tion, prediction, identification or assessment of a therapy as referred to herein) can be made for a statistically significant portion of subjects. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98%, or at least 99%. The p-values are, preferably, 0.4, 0.1, 0.05, 0.01, 0.005, or 0.0001.

In accordance with the method of the present invention, the risk of stroke shall be predicted. The term “stroke” is well known in the art. The term, preferably, refers to ischemic stroke, in particular to cerebral ischemic stroke. A stroke which is predicted by the method of the present invention shall be caused by reduced blood flow to the brain or parts thereof which leads to an undersupply of oxygen to brain cells. In particular, the stroke leads to irreversible tissue damage due to brain cell death. Symptoms of stroke are well known in the art. Ischemic stroke may be caused by atherothrombosis or embolism of a major cerebral artery, by coagulation disorders or nonatheromatous vascular disease, or by cardiac ischemia which leads to a reduced overall blood flow. The ischemic stroke is preferably selected from the group consisting of atherothrombotic stroke, cardioembolic stroke and lacunar stroke. Preferably, the stroke to be predicted is an acute ischemic stroke, in particular cardioembolic stroke. A cardioembolic stroke (frequently also referred to as embolic or thromboembolic stroke) can be caused by atrial fibrillation.

The term “stroke” does, preferably, not include hemorrhagic stroke. Whether a subject suffers from stroke, in particular from ischemic stroke can be determined by well-known methods. Moreover, symptoms of stroke are well known in the art. E.g., stroke symptoms include sudden numbness or weakness of face, arm or leg, especially on one side of the body, sudden confusion, trouble speaking or understanding, sudden trouble seeing in one or both eyes, and sudden trouble walking, dizziness, loss of balance or coordination.

It is known in the art that biomarkers could be altered in various diseases and disorders. This does also apply to CES-2. Accordingly, the expression “prediction of the risk of stroke” as an aid in the prediction of a risk of an adverse event associated with atrial fibrillation.

The “subject” as referred to herein is, preferably, a mammal. Mammals include, but are not limited to, domesticated animals (e.g., cows, sheep, cats, dogs, and horses), primates (e.g., humans and non-human primates such as monkeys), rabbits, and rodents (e.g., mice and rats). Preferably, the subject is a human subject.

Preferably, the subject to be tested is of any age, more preferably, the subject to be tested is 50 years of age or older, more preferably 60 years of age or older, and most preferably 65 years of age or older. Further, it is envisaged that the subject to be tested is 70 years of age or older. Moreover, it is envisaged that the subject to be tested is 75 years of age or older. Also, the subject may be between 50 and 90 years.

Further, the age of the subject may be 55 years or older.

In a preferred embodiment of the method of the present invention, the subject to be tested suffers from atrial fibrillation. Atrial fibrillation may be paroxysmal, persistent or permanent atrial fibrillation. Thus, the subject may suffer from paroxysmal, persistent or permanent atrial fibrillation. In particular, it is envisaged that the subject suffers from paroxysmal, persistent or permanent atrial fibrillation. It has been shown in the studies underlying the present invention that the determination of the biomarkers as referred to herein allows for a reliable prediction of stroke in all subgroups.

Thus, in an embodiment of the present invention, the subject suffers from paroxysmal atrial fibrillation.

In another embodiment of the present invention, the subject suffers from persistent atrial fibrillation.

The term “Atrial Fibrillation” is well known in the art. As used herein, the term preferably refers to a supraventricular tachyarrhythmia characterized by uncoordinated atrial activation with consequent deterioration of atrial mechanical function. In particular, the term refers to an abnormal heart rhythm characterized by rapid and irregular beating. It involves the two upper chambers of the heart. In a normal heart rhythm, the impulse generated by the sino-atrial node spreads through the heart and causes contraction of the heart muscle and pumping of blood. In atrial fibrillation, the regular electrical impulses of the sino-atrial node are re-placed by disorganized, rapid electrical impulses which result in irregular heartbeats. Symptoms of atrial fibrillation are heart palpitations, fainting, shortness of breath, or chest pain. However, most episodes have no symptoms. On the electrocardiogram atrial fibrillation is characterized by the replacement of consistent P waves by rapid oscillations or fibrillatory waves that vary in amplitude, shape, and timing, associated with an irregular, frequently rapid ventricular response when atrioventricular conduction is intact.

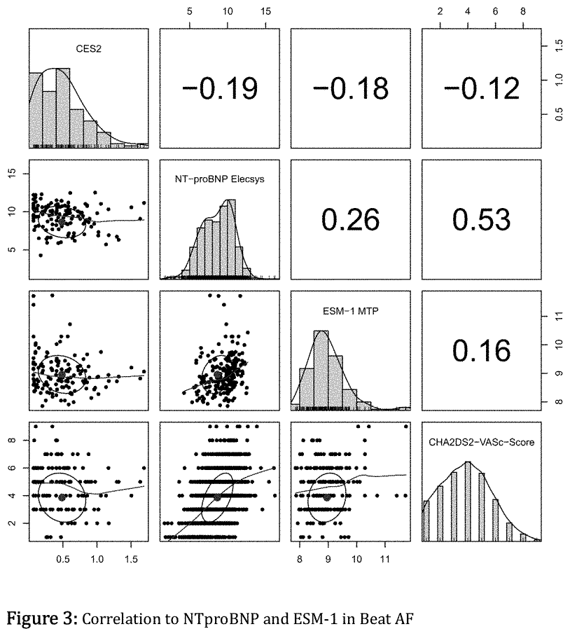

The American College of Cardiology (ACC), American Heart Association (AHA), and the European Society of Cardiology (ESC) propose the following classification system (see Fuster (2006) Circulation 114 (7): e257-354 which herewith is incorporated by reference in its entirety, see e.g. FIG. 3 in the document): First detected AF, paroxysmal AF, persistent AF, and permanent AF.

All people with AF are initially in the category called first detected AF. However, the subject may or may not have had previous undetected episodes. A subject suffers from permanent AF, if the AF has persisted for more than one year. In particular, conversion back to sinus rhythm does not occur (or only with medical intervention). A subject suffers from persistent AF, if the AF lasts more than 7 days. The subject may require either pharmacologic or electrical intervention to terminate atrial fibrillation. Thus persistent AF occurs in episodes, but the arrhythmia does typically not convert back to sinus rhythm spontaneously (i.e. without medical invention). Paroxysmal atrial fibrillation, preferably, refers to an intermittent episode of atrial fibrillation which lasts not longer than 7 days and terminates spontaneously (i.e. without medical intervention). In most cases of paroxysmal AF, the episodes last less than 24 hours. Thus, whereas paroxysmal atrial fibrillation terminates spontaneously, persistent atrial fibrillation does not end spontaneously and requires electrical or pharmacological cardioversion for termination, or other procedures, such as ablation procedures (Fuster (2006) Circulation 114 (7): e257-354). The term “paroxysmal atrial fibrillation” is defined as episodes of AF that terminate spontaneously in less than 48 hours, more preferably in less than 24 hours, and, most preferably in less than 12 hours. Both persistent and paroxysmal AF may be recurrent.

As set forth above, the subject to be tested preferably suffers from paroxysmal, persistent or permanent atrial fibrillation.

Further, it is envisaged that the subject suffers from an episode of atrial fibrillation at the time when the sample is obtained. This may be e.g. the case if the subject suffers from permanent or persistent AF.

Alternatively, it is envisaged that the subject does not suffer from an episode of atrial fibrillation at the time when the sample is obtained. This may be e.g. the case if the subject suffers from paroxysmal AF. Accordingly, the subject shall have a normal sinus rhythm when the sample is obtained, i.e. is in sinus rhythm.

Further, it is contemplated that the atrial fibrillation has been diagnosed previously in the subject. Accordingly, the atrial fibrillation shall be a diagnosed, i.e. a detected, atrial fibrillation.

As shown in the Examples, a prediction of the risk is possible in patients with heart failure.

Accordingly, the subject to be tested may suffer from heart failure. The term “heart failure” in accordance with the method of the present invention preferably relates to heart failure with reduced left ventricular ejection fraction.

Further, it has been shown that a prediction of the risk is possible in subjects who do not have a history of heart failure. Accordingly, the subject to be tested preferably does not suffer from heart failure. In particular, the subject to be tested does not suffer from heart failure according to NYHA class II, III, and IV.

In particular, preferred embodiment, the subject is a subject who does not suffer from heart failure, but suffers from atrial fibrillation.

Advantageously, it has been shown in the studies underlying the method of the present invention that a reliable prediction is possible even if the subject already receives anticoagulation therapy, i.e. a therapy which aims to reduce the risk of stroke (about 70% of patients received received oral anticoagulation and about 30% vitamin K antagonists such as warfarin and dicumarol). Surprisingly, it has been shown that by determining the amounts of CES-2 could be differentiated within a population or risk patient, i.e. patients with atrial fibrillation receiving anticoagulation therapy, it could be reliably differentiated between a reduced risk and an increased risk of stroke. AF patients with an increased risk of stroke might benefit from an intensification of anticoagulation therapy. Moreover, AF patients which a reduced risk of stroke might be overtreated and might benefit from a less intense anticoagulation therapy (resulting, e.g., in decreased health care costs).

Thus, it is preferred in accordance with the present invention that the subject receives anticoagulation therapy.

As set forth above, anticoagulation therapy is preferably a therapy which aims to reduce the risk of anticoagulation in said subject. More preferably, anticoagulation therapy is the administration of at least one anticoagulant. Administration of at least one anticoagulant shall aim to reduce or prevent coagulation of blood and related stroke. In a preferred embodiment, at least one anticoagulant is selected from the group consisting of heparin, a coumarin derivative (i.e. a vitamin K antagonist), in particular warfarin or dicumarol, oral anticoagulants, in particular dabigatran, rivaroxaban or apixaban, tissue factor pathway inhibitor (TFPI), antithrombin III, factor IXa inhibitors, factor Xa inhibitors, inhibitors of factors Va and VIIIa and thrombin inhibitors (anti-IIa type). Accordingly, it is envisaged that the subject takes at least one of the aforementioned medicaments.

In preferred embodiment, the anticoagulant is a vitamin K antagonist such as warfarin or dicumarol. Vitamin K antagonists, such as warfarin or dicumarol are less expensive, but need better patient compliance, because of the inconvenient, cumbersome and often unreliable treatment with fluctuating time in therapeutic range. NOAC (new oral anticoagulants) comprise direct factor Xa inhibitors (apixaban, rivaroxaban, darexaban, edoxaban), direct thrombin inhibitors (dabigatran) and PAR-1 antagonists (vorapaxar, atopaxar).

In another preferred embodiment the anticoagulant and oral anticoagulant, in particular apixaban, rivaroxaban, darexaban, edoxaban, dabigatran, vorapaxar, or atopaxar.

Thus, the subject to be tested may be on therapy with an oral anticoagulant or a vitamin K antagonist at the time of the testing (i.e. at the time at which the sample is received.

In a preferred embodiment, the method for predicting the risk of stroke in a subject further comprises i) the step of recommending anticoagulation therapy, or ii) of recommending an intensification of anticoagulation therapy, if the subject has been identified to be at risk to suffer from stroke. In a preferred embodiment, the method for predicting the risk of stroke in a subject further comprises i) the step of initiating anticoagulation therapy, or ii) of intensifying anticoagulation therapy, if the subject has been identified to be at risk to suffer from stroke (by the method of the present invention).

The term “recommending” as used herein means establishing a proposal for a therapy which could be applied to the subject. However, it is to be understood that applying the actual therapy whatsoever is not comprised by the term. The therapy to be recommended depends on the outcome of the provided by the method of the present invention.

In particular, the following applies:

If the subject to be tested does not receive anticoagulation therapy, the initiation of anticoagulation is recommended, if the subject has been identified to be at risk to suffer from stroke. Thus, anticoagulation therapy shall be initiated.

If the subject to be tested already receives anticoagulation therapy, the intensification of anticoagulation is recommended, if the subject has been identified to be at risk to suffer from stroke. Thus, anticoagulation therapy shall be intensified.

In a preferred embodiment, anticoagulation therapy is intensified by increasing the dosage of the anticoagulant, i.e. the dosage of the currently administered coagulant.

In a particularly preferred embodiment, anticoagulation therapy is intensified by increasing the replacing the currently administered anticoagulant with a more effective anticoagulant. Thus, a replacement of the anticoagulant is recommended.

The method of the present invention can be used for assessing the efficacy of an anticoagulation therapy of a subject, by determining the amount of CES-2 and optionally one or more biomarkers comprising of a natriuretic peptide, ESM-1, ANG-2, IGFBP7 in a sample from the subject, and by comparing the amount of CES-2 and optionally one or more biomarkers comprising of a natriuretic peptide, ESM-1, ANG-2, IGFBP7 to a reference amount (or to reference amounts), whereby the risk of stroke is to be assessed.

In a preferred embodiment of the present invention, a decreased amount of CES-2 is significant that anticoagulation therapy is not efficient, and wherein a normal or an increased amount of CES-2 is significant that anticoagulation therapy is effective.

If the subject to be tested receives an anticoagulation therapy and has a decreased amount of CES-2, the intensification of anticoagulation is recommended. Thus, anticoagulation therapy shall be intensified or a replacement of the anticoagulant is recommended.

The present invention further concerns a method for identifying a subject being eligible to the administration of at least one anticoagulation medicament or being eligible for increasing the dosage of at least one anticoagulation medicament, comprising a) determining the amount CES-2 and optionally one or more biomarkers comprising of a natriuretic peptide, ESM-1, ANG-2, IGFBP7 and b) comparing the amount as determined in step a) with a reference amount, whereby a subject being eligible to the administration of said at least one medicament or to an increased dosage of said at least one medicament is identified.

The present invention further relates to a method for monitoring a subject receiving an anticoagulation therapy, comprising the steps of a) determining the amount of CES-2 and optionally one or more biomarkers comprising of a natriuretic peptide, ESM-1, ANG-2, IGFBP7 in a sample from the subject, and b) comparing the amount of CES-2 and optionally of one or more biomarkers comprising of a natriuretic peptide, ESM-1, ANG-2, IGFBP7 to a reference amount (or to reference amounts), whereby the risk of stroke is to be assessed.

Accordingly, by carrying out the method of the present invention a subject can be identified who requires closer monitoring, in particular with respect to the anticoagulation therapy (and, thus, closer observation). With “closer monitoring” it is, preferably, meant that biomarkers as referred herein, i.e. the CES-2 and one or more biomarkers comprising of a natriuretic peptide, ESM-1, ANG-2, IGFBP7 are determined in at least one further sample obtained from the subject after a short interval after the sample referred to in step a) of the method of the present invention.

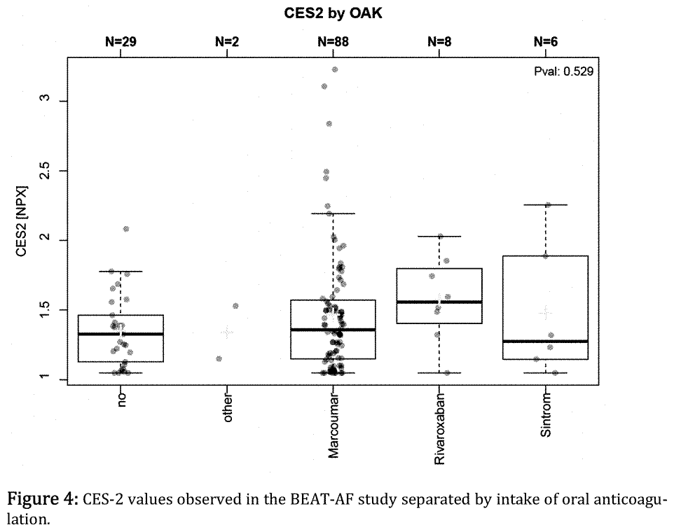

It has been described that better prevention in high risk patients is achieved with the oral anticoagulant apixaban versus the vitamin K antagonist warfarin as shown in Hijazi at al., The Lancet 2016 387, 2302-2311, (FIG. 4).

Thus, it is envisaged that the subject to be tested is a subject who is treated with a vitamin K antagonist such as warfarin or dicumarol. If the subject has been identified to be at risk to suffer from stroke (by the method of the present invention, it the replacement of the vitamin K antagonist with an oral anticoagulant, in particular dabigatran, rivaroxaban or apixaban is recommended. According the therapy with the vitamin K antagonist is discontinued and therapy with an oral anticoagulant is initiated.

In a preferred embodiment of the present invention, the subject has a history of stroke or TIA (transient ischemic attack). In particular, the subject has a history of stroke.

Accordingly, it is envisaged that the subject has suffered from stroke or TIA prior to carrying out the method of the present invention (or to be more precise prior to obtaining the sample to be tested). Although the subject shall have suffered from stroke or TIA in the past, the subject shall not suffer from stroke and TIA at the time at which the sample to be tested is obtained).

As set forth above, the biomarker CES-2 could be altered in various diseases and disorders other than atrial fibrillation. In an embodiment of the present invention, it is envisaged that the subject does not suffer from such diseases and disorders.

The method of the present invention can be also used for the screening of larger populations of subjects. Therefore, it is envisaged, that at least 100 subjects, in particular at least 1000 subjects are assessed with respect to the risk of stroke Thus, the amount of the biomarker is determined in samples from at least 100, or in particular of from at least 1000 subjects. Moreover, it is envisaged that at least 10.000 subjects are assessed.

The term “sample” refers to a sample of a body fluid, to a sample of separated cells or to a sample from a tissue or an organ. Samples of body fluids can be obtained by well-known techniques and include, samples of blood, plasma, serum, urine, lymphatic fluid, sputum, ascites, or any other bodily secretion or derivative thereof. Tissue or organ samples may be obtained from any tissue or organ by, e.g., biopsy. Separated cells may be obtained from the body fluids or the tissues or organs by separating techniques such as centrifugation or cell sorting. E.g., cell-, tissue- or organ samples may be obtained from those cells, tissues or organs which express or produce the biomarker. The sample may be frozen, fresh, fixed (e.g. formalin fixed), centrifuged, and/or embedded (e.g. paraffin embedded), etc. The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the biomarker(s) in the sample.

In a preferred embodiment of the present invention, the sample is a blood (i.e. whole blood), serum or plasma sample. Serum is the liquid fraction of whole blood that is obtained after the blood is allowed to clot. For obtaining the serum, the clot is removed by centrifugation and the supernatant is collected. Plasma is the acellular fluid portion of blood. For obtaining a plasma sample, whole blood is collected in anticoagulant-treated tubes (e.g. citrate-treated or EDTA-treated tubes). Cells are removed from the sample by centrifugation and the supernatant (i.e. the plasma sample) is obtained.

Preferably, the term “predicting the risk” as used herein refers to assessing the probability according to which the subject will suffer of stroke. Typically, it is predicted whether a subject is at risk (and thus at elevated risk) or not at risk (and thus at reduced risk) of suffering from stroke. Accordingly, the method of the present invention allows for differentiating between a subject who is at risk of stroke and a subject who is not at risk of stroke. Further, it is envisaged that the method of the present invention allows for differentiating between a reduced, average, and elevated risk of stroke.

As set forth above, the risk (and probability) of suffering from stroke within a certain time window shall be predicted. In accordance with the present invention, it is envisaged that the short term risk or the long risk is predicted. E.g., the risk to suffer from stroke within one week or within one month is predicted. The shortest timespan observed in the studies underlying the present invention was 11 days. The subject had decreased levels of CES-2. This indicates that not only a long term but also a short term prediction is possible.

In an embodiment of the present invention, the predictive window is a period of about at least three months, about at least six months, or about at least one year. In another preferred embodiment, the predictive window is a period of about five years. Further, the predictive window might be a period of about six years (e.g. for the prediction of stroke).

In an embodiment, the predictive window is a period of up to 10 years. Thus, the risk to suffer from stroke within ten years is predicted.

In another embodiment, the predictive window is a period of up to 7 years. Thus, the risk to suffer from stroke within seven years is predicted.

In another embodiment, the predictive window is a period of up to 3 years. Thus, the risk to suffer from stroke within three years is predicted.

Also, it is envisaged that the predictive window a period of 1 to 10 years.

Preferably, the predictive window is calculated from the completion of the method of the present invention. More preferably, said predictive window is calculated from the time point at which the sample to be tested has been obtained.

As set forth above, the expression “predicting the risk of stroke” means that the subject to be analyzed by the method of the present invention is allocated either into the group of subjects being at risk of suffering from stroke, or into the group of subjects not being at risk of suffering from stroke. Thus, it is predicted whether the subject is at risk or not at risk of suffering from stroke. As used herein “a subject who is at risk of suffering from stroke”, preferably has an elevated risk of suffering from stroke (preferably within the predictive window). Preferably, said risk is elevated as compared to the average risk in a cohort of subjects. As used herein, “a subject who is not at risk of suffering from stroke”, preferably, has a reduced risk of suffering from stroke (preferably within the predictive window). Preferably, said risk is reduced as compared to the average risk in a cohort of subjects. A subject who is at risk of suffering from stroke preferably has a risk of suffering from stroke of at least 7% or more preferably of at least 10%, preferably, within a predictive window of five years. A subject who is not at risk of suffering from stroke preferably has a risk of lower than 5%, more preferably of lower than 3% of suffering from stroke, preferably within a predictive window of five years.

The biomarker Carboxylesterase-2 (abbreviated CES-2) is well known in the art. The biomarker is frequently also referred to as CES-2; iCE; CE-2; PCE-2; CES-2A1. CES-2 is predominantly expressed in the small intestine. Furthermore, it is expressed among others in heart, brain, testis, skeletal muscle, colon, spleen, kidney and liver.

In a preferred embodiment of the present invention, the amount of the human CES-2 polypeptide is determined in a sample from the subject. The sequence of the human CES-2 polypeptide is well known in the art and can be e.g. assessed via Uniprot database, see entry (UniProtKB—000748 (EST2_HUMAN).

The human CES-2 gene locates on chromosome 16. CES-2 is a protein of around 60 kDa polypeptides, alternative splicing results in multiple variants encoding the same protein. 12 transcripts (splice variants), 130 orthologues, 12 paralogues, and 4 phenotypes of the gene were described. Furthermore, CES-2 is associated with 4 phenotypes. CES-2 contains 12 (15) exons. (Ensembl release 93, http://www.ensembl.org).

Wu et al., (Pharmacogenetics. 2003 July; 13(7):425-35) identified three different promoters, wherein two promoters are tissue specific and a further distal promoter is responsible for low level expression of the gene in many tissues.

CES-2 gene is transcribed into 12 different isoforms, only half of them are protein coding (https://www.ncbi.nlm nih.gov/gene/8824#reference-sequences).

In a preferred embodiment, the amount of isoform 1 of the CES-2 transcript is determined, i.e. isoform 1 having a sequence of 623 amino acids as shown under RefSeq accession number NP_003860.2.

In a preferred embodiment, the amount of isoform 2 of the CES-2 transcript is determined, i.e. isoform 2 having a sequence of 607 amino acids as shown under RefSeq accession number NP_932327.1.

In a preferred embodiment, the amount of isoform 3 of the CES-2 transcript is determined, i.e. isoform 3 having a sequence of 450 amino acids as shown under RefSeq accession number XP_016879307.1.

In a preferred embodiment, the amount of isoform 4 of the CES-2 transcript is determined, i.e. isoform 4 having a sequence of 466 amino acids as shown under RefSeq accession number XP_011521723.1.

In another preferred embodiment, the amount of isoform 1, 2, 3, 4, 5, 6, 7, 8, 9, 10, 11 and 12 of the CES-2 transcript is determined, i.e. total CES-2.

For example, the amount of CES-2 could be determined with a monoclonal antibody (such as a mouse antibody) against amino acids of the CES-2 polypeptide and/or with a goat polyclonal antibody.

In another preferred embodiment CES-2 is determined in combination with a natriuretic peptide and/or with ESM1.

The term “natriuretic peptide” comprises atrial natriuretic peptide (ANP)-type and brain natriuretic peptide (BNP)-type peptides. Thus, natriuretic peptides according to the present invention comprise ANP-type and BNP-type peptides and variants thereof (see, e.g., Bonow R O. et al., Circulation 1996; 93: 1946-1950).

ANP-type peptides comprise pre-proANP, proANP, NT-proANP, and ANP.

BNP-type peptides comprise pre-proBNP, proBNP, NT-proBNP, and BNP.

The pre-pro peptide (134 amino acids in the case of pre-proBNP) comprises a short signal peptide, which is enzymatically cleaved off to release the pro peptide (108 amino acids in the case of proBNP). The pro peptide is further cleaved into an N-terminal pro peptide (NT-pro peptide, 76 amino acids in case of NT-proBNP) and the active hormone (32 amino acids in the case of BNP, 28 amino acids in the case of ANP).

Preferred natriuretic peptides according to the present invention are NT-proANP, ANP, NT-proBNP, BNP. ANP and BNP are the active hormones and have a shorter half-life than their respective inactive counterparts, NT-proANP and NT-proBNP. BNP is metabolized in the blood, whereas NT-proBNP circulates in the blood as an intact molecule and as such is eliminated renally

Preanalytics are more robust with NT-proBNP, allowing easy transportation of the sample to a central laboratory (Mueller T, Gegenhuber A, Dieplinger B, Poelz W, Haltmayer M. Long-term stability of endogenous B-type natriuretic peptide (BNP) and amino terminal proBNP (NT-proBNP) in frozen plasma samples. Clin Chem Lab Med 2004; 42: 942-4.). Blood samples can be stored at room temperature for several days or may be mailed or shipped without recovery loss. In contrast, storage of BNP for 48 hours at room temperature or at 4° C. leads to a concentration loss of at least 20% (Mueller T, Gegenhuber A, et al., Clin Chem Lab Med 2004; 42: 942-4; Wu A H, Packer M, Smith A, Bijou R, Fink D, Mair J, Wallentin L, Johnston N, Feldcamp C S, Haverstick D M, Ahnadi C E, Grant A, Despres N, Bluestein B, Ghani F. Analytical and clinical evaluation of the Bayer ADVIA Centaur automated B-type natriuretic peptide assay in patients with heart failure: a multisite study. Clin Chem 2004; 50: 867-73.). Therefore, depending on the time-course or properties of interest, either measurement of the active or the inactive forms of the natriuretic peptide can be advantageous.

The most preferred natriuretic peptides according to the present invention are NT-proBNP and BNP, in particular NT-proBNP. As briefly discussed above, the human NT-proBNP as referred to in accordance with the present invention is a polypeptide, comprising preferably, 76 amino acids in length corresponding to the N-terminal portion of the human NT-proBNP molecule. The structure of the human BNP and NT-proBNP has been described already in detail in the prior art, e.g., WO 02/089657, WO 02/083913, and Bonow R O. Et al., New Insights into the cardiac natriuretic peptides. Circulation 1996; 93: 1946-1950. Preferably, human NT-proBNP as used herein is human NT-proBNP as disclosed in EP 0 648 228 B1.

The term “ESM1” also named Endocan, comprises is a proteoglycan composed of a 20 kDa mature polypeptide and a 30 kDa O-linked glycan chain and variants thereof (Bechard D et al., J Biol Chem 2001; 276(51):48341-48349)

In a preferred embodiment of the present invention, the amount of the human ESM-1 poly-peptide is determined in a sample from the subject. The sequence of the human ESM-1 polypeptide is well known in the art (see e.g. Lassale P. et al., J. Biol. Chem. 1996; 271:20458-20464 and can be e.g. assessed via Uniprot database, see entry Q9NQ30 (ESM1_HUMAN). Two isoforms of ESM-1 are produced by alternative splicing, isoform 1 (having the Uniprot identifier Q9NQ30-1) and isoform 2 (having the Uniprot identifier Q9NQ30-2). Isoform 1 has length of 184 amino acids. In isoform 2, amino acids 101 to 150 of isoform 1 are missing. Amino acids 1 to 19 form the signal peptide (which might be cleaved off).

In a preferred embodiment, the amount of isoform 1 of the ESM-1 polypeptide is determined, i.e. isoform 1 having a sequence as shown under UniProt accession number Q9NQ30-1.

In another preferred embodiment, the amount of isoform 2 of the ESM-1 polypeptide is determined, i.e. isoform 2 having a sequence as shown under UniProt accession number Q9NQ30-2.

In another preferred embodiment, the amount of isoform-1 and isoform 2 of the ESM-1 polypeptide is determined, i.e. total ESM-1.

For example, the amount of ESM-1 could be determined with a monoclonal antibody (such as a mouse antibody) against amino acids 85 to 184 of the ESM-1 polypeptide and/or with a goat polyclonal antibody.

The biomarker Angiopoietin-2 (abbreviated “Ang-2”, frequently also referred to as ANGPT2) is well known in the art. It is a naturally occurring antagonist for both Ang-1 and TIE2 (see e.g. Maisonpierre et al., Science 277 (1997) 55-60). The protein can induce tyrosine phosphorylation of TEK/TIE2 in the absence of ANG-1. In the absence of angiogenic inducers, such as VEGF, ANG2-mediated loosening of cell-matrix contacts may induce endothelial cell apoptosis with consequent vascular regression. In concert with VEGF, it may facilitate endothelial cell migration and proliferation, thus serving as a permissive angiogenic signal. The sequence of human Angiopoietin is well known in the art. Uniprot lists three isoforms of Angiopoietin-2: Isoform 1 (Uniprot identifier: 015123-1), Isoform 2 (identifier: 015123-2) and Isoform 3 (015123-3). In a preferred embodiment, the total amount of Angiopoietin-2 is determined. The total amount is preferably the sum of the amounts of complexed and free Angiopoietin-2.

IGFBP-7 (Insulin-like Growth Factor Binding Protein 7) is a 30-kDa modular glycoprotein known to be secreted by endothelial cells, vascular smooth muscle cells, fibroblasts, and epithelial cells (Ono, Y., et al., Biochem Biophys Res Comm 202 (1994) 1490-1496). Preferably, the term “IGFBP-7” refers to human IGFBP-7. The sequence of the protein is well-known in the art and is e.g. accessible via UniProt (Q16270, IBP7_HUMAN), or via Gen-Bank (NP_001240764.1). A detailed definition of the biomarker IGFBP-7 is e.g. provided in WO 2008/089994 which herewith is incorporated by reference in its entirety. There are two isoforms of IGFBP-7, Isoform 1 and 2 which are produced by alternative splicing. In an embodiment of the present invention, the total amount of both isoforms is measured (for the sequence, see the UniProt database entry (Q16270-1 and Q16270-2).

The term “determining” the amount of a biomarker as referred to herein (such as CES-2 or the natriuretic peptide) refers to the quantification of the biomarker, e.g. to measuring the level of the biomarker in the sample, employing appropriate methods of detection described elsewhere herein. The terms “measuring” and “determining” are used herein interchangeably.

In an embodiment, the amount of a biomarker is determined by contacting the sample with an agent that specifically binds to the biomarker, thereby forming a complex between the agent and said biomarker, detecting the amount of complex formed, and thereby measuring the amount of said biomarker.

The biomarkers as referred to herein (such as CES-2) can be detected using methods generally known in the art. Methods of detection generally encompass methods to quantify the amount of a biomarker in the sample (quantitative method). It is generally known to the skilled artisan which of the following methods are suitable for qualitative and/or for quantitative detection of a biomarker. Samples can be conveniently assayed for, e.g., proteins using Westerns and immunoassays, like ELISAs, RIAs, fluorescence- and luminescence-based immunoassays and proximity extension assays, which are commercially available. Further suitable methods to detect biomarkers include measuring a physical or chemical property specific for the peptide or polypeptide such as its precise molecular mass or NMR spectrum. Said methods comprise, e.g., biosensors, optical devices coupled to immunoassays, biochips, analytical devices such as mass-spectrometers, NMR—analyzers, or chromatography devices. Further, methods include microplate ELISA-based methods, fully-automated or robotic immunoassays (available for example on Elecsys™ analyzers), CBA (an enzymatic Cobalt Binding Assay, available for example on Roche-Hitachi™ analyzers), and latex agglutination assays (available for example on Roche-Hitachi™ analyzers).

For the detection of biomarker proteins as referred to herein a wide range of immunoassay techniques using such an assay format are available, see, e.g., U.S. Pat. Nos. 4,016,043, 4,424,279, and 4,018,653. These include both single-site and two-site or “sandwich” assays of the non-competitive types, as well as in the traditional competitive binding assays. These assays also include direct binding of a labeled antibody to a target biomarker.

Methods employing electrochemiluminescent labels are well-known. Such methods make use of the ability of special metal complexes to achieve, by means of oxidation, an excited state from which they decay to ground state, emitting electrochemiluminescence. For review see Richter, M. M., Chem. Rev. 2004; 104: 3003-3036.

In an embodiment, the detection antibody (or an antigen-binding fragment thereof) to be used for measuring the amount of a biomarker is ruthenylated or iridinylated. Accordingly, the antibody (or an antigen-binding fragment thereof) shall comprise a ruthenium label. In an embodiment, said ruthenium label is a bipyridine-ruthenium (II) complex. Or the antibody (or an antigen-binding fragment thereof) shall comprise an iridium label. In an embodiment, said iridium label is a complex as disclosed in WO 2012/107419.

In an embodiment of the sandwich assay for the determination of CES-2, the assay comprises a biotinylated first monoclonal antibody that specifically binds CES-2 (as capture antibody) and a ruthenylated F(ab′)2-fragment of a second monoclonal antibody that specifically binds CES-2 as detection antibody). The two antibodies form sandwich immunoassay complexes with CES-2 in the sample.

In an embodiment of the sandwich assay for the determination of the natriuretic peptide, the assay comprises a biotinylated first monoclonal antibody that specifically binds the natriuretic peptide (as capture antibody) and a ruthenylated F(ab′)2-fragment of a second monoclonal antibody that specifically binds the natriuretic peptide as detection antibody). The two antibodies form sandwich immunoassay complexes with the natriuretic peptide in the sample.

Measuring the amount of a polypeptide (such as CES-2 or the natriuretic peptide natriuretic peptide, ESM-1, ANG-2, IGFBP7) may, preferably, comprise the steps of (a) contacting the polypeptide with an agent that specifically binds said polypeptide, (b) (optionally) removing non-bound agent, (c) measuring the amount of bound binding agent, i.e. the complex of the agent formed in step (a). According to a preferred embodiment, said steps of contacting, removing and measuring may be performed by an analyzer unit. According to some embodiments, said steps may be performed by a single analyzer unit of said system or by more than one analyzer unit in operable communication with each other. For example, according to a specific embodiment, said system disclosed herein may include a first analyzer unit for performing said steps of contacting and removing and a second analyzer unit, operably connected to said first analyzer unit by a transport unit (for example, a robotic arm), which performs said step of measuring.

The agent which specifically binds the biomarker (herein also referred to as “binding agent”) may be coupled covalently or non-covalently to a label allowing detection and measurement of the bound agent. Labeling may be done by direct or indirect methods. Direct labeling involves coupling of the label directly (covalently or non-covalently) to the binding agent. Indirect labeling involves binding (covalently or non-covalently) of a secondary binding agent to the first binding agent. The secondary binding agent should specifically bind to the first binding agent. Said secondary binding agent may be coupled with a suitable label and/or be the target (receptor) of a tertiary binding agent binding to the secondary binding agent. Suitable secondary and higher order binding agents may include antibodies, secondary antibodies, and the well-known streptavidin-biotin system (Vector Laboratories, Inc.). The binding agent or substrate may also be “tagged” with one or more tags as known in the art. Such tags may then be targets for higher order binding agents. Suitable tags include biotin, digoxygenin, His-Tag, Glutathion-S-Transferase, FLAG, GFP, myc-tag, influenza A virus haemagglutinin (HA), maltose binding protein, and the like. In the case of a peptide or polypeptide, the tag is preferably at the N-terminus and/or C-terminus. Suitable labels are any labels detectable by an appropriate detection method. Typical labels include gold particles, latex beads, acridan ester, luminol, ruthenium complexes, iridium complexes, enzymatically active labels, radioactive labels, magnetic labels (“e.g. magnetic beads”, including paramagnetic and superparamagnetic labels), and fluorescent labels. Enzymatically active labels include e.g. horseradish peroxidase, alkaline phosphatase, beta-Galactosidase, Luciferase, and derivatives thereof. Suitable substrates for detection include di-amino-benzidine (DAB), 3,3′-5,5′-tetramethylbenzidine, NBT-BCIP (4-nitro blue tetrazolium chloride and 5-bromo-4-chloro-3-indolyl-phosphate, avail-able as ready-made stock solution from Roche Diagnostics), CDP-Star™ (Amersham Bio-sciences), ECF™ (Amersham Biosciences). A suitable enzyme-substrate combination may result in a colored reaction product, fluorescence or chemoluminescence, which can be determined according to methods known in the art (e.g. using a light-sensitive film or a suit-able camera system). As for measuring the enzymatic reaction, the criteria given above apply analogously. Typical fluorescent labels include fluorescent proteins (such as GFP and its derivatives), Cy3, Cy5, Texas Red, Fluorescein, and the Alexa dyes (e.g. Alexa 568). Further fluorescent labels are available e.g. from Molecular Probes (Oregon). Also the use of quantum dots as fluorescent labels is contemplated. A radioactive label can be detected by any method known and appropriate, e.g. a light-sensitive film or a phosphor imager.

The amount of a polypeptide may be, also preferably, determined as follows: (a) contacting a solid support comprising a binding agent for the polypeptide as described elsewhere herein with a sample comprising the peptide or polypeptide and (b) measuring the amount of peptide or poly-peptide which is bound to the support. Materials for manufacturing supports are well-known in the art and include, inter alia, commercially available column materials, polystyrene beads, latex beads, magnetic beads, colloid metal particles, glass and/or silicon chips and surfaces, nitrocellulose strips, membranes, sheets, duracytes, wells and walls of reaction trays, plastic tubes etc.

In yet an aspect the sample is removed from the complex formed between the binding agent and the at least one marker prior to the measurement of the amount of formed complex. Accordingly, in an aspect, the binding agent may be immobilized on a solid support. In yet an aspect, the sample can be removed from the formed complex on the solid support by applying a washing solution.

“Sandwich assays” are among the most useful and commonly used assays encompassing a number of variations of the sandwich assay technique. Briefly, in a typical assay, an unlabeled (capture) binding agent is immobilized or can be immobilized on a solid substrate, and the sample to be tested is brought into contact with the capture binding agent. After a suitable period of incubation, for a period of time sufficient to allow formation of a binding agent-biomarker complex, a second (detection) binding agent labeled with a reporter molecule capable of producing a detectable signal is then added and incubated, allowing time sufficient for the formation of another complex of binding agent-biomarker-labeled binding agent. Any unreacted material may be washed away, and the presence of the biomarker is determined by observation of a signal produced by the reporter molecule bound to the detection binding agent. The results may either be qualitative, by simple observation of a visible signal, or may be quantitated by comparison with a control sample containing known amounts of biomarker.

The incubation steps of a typical sandwich assays can be varied as required and appropriate. Such variations include for example simultaneous incubations, in which two or more of binding agent and biomarker are co-incubated. For example, both, the sample to be analyzed and a labeled binding agent are added simultaneously to an immobilized capture binding agent. It is also possible to first incubate the sample to be analyzed and a labeled binding agent and to thereafter add an antibody bound to a solid phase or capable of binding to a solid phase.

The formed complex between a specific binding agent and the biomarker shall be proportional to the amount of the biomarker present in the sample. It will be understood that the specificity and/or sensitivity of the binding agent to be applied defines the degree of proportion of at least one marker comprised in the sample which is capable of being specifically bound. Further details on how the measurement can be carried out are also found elsewhere herein. The amount of formed complex shall be transformed into an amount of the biomarker reflecting the amount indeed present in the sample.

The terms “binding agent”, “specific binding agent”, “analyte-specific binding agent”, “detection agent” and “agent that specifically binds to a biomarker” are used interchangeably herein. Preferably it relates to an agent that comprises a binding moiety which specifically binds the corresponding biomarker. Examples of “binding agents”, “detection agents”, “agents” are a nucleic acid probe, nucleic acid primer, DNA molecule, RNA molecule, aptamer, antibody, antibody fragment, peptide, peptide nucleic acid (PNA) or chemical compound. A preferred agent is an antibody which specifically binds to the biomarker to be determined. The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity (i.e. antigen-binding fragments thereof). Preferably, the antibody is a polyclonal antibody (or an antigen-binding fragment therefrom). More preferably, the antibody is a monoclonal antibody (or an antigen binding fragment therefore Moreover, as described elsewhere herein, it is envisaged that two monoclonal antibodies are used that bind at different positions of CES-2 (in a sandwich immunoassay). Thus, at least one antibody is used for the determination of the amount of CES-2.

In an embodiment, the at least one antibody is a mouse monoclonal antibody. In another embodiment, the at least one antibody is a rabbit monoclonal antibody. In a further embodiment, the antibody is goat polyclonal antibody. In an even further embodiment, the antibody is a sheep polyclonal antibody.

The term “specific binding” or “specifically bind” refers to a binding reaction wherein binding pair molecules exhibit a binding to each other under conditions where they do not significantly bind to other molecules. The term “specific binding” or “specifically binds”, when referring to a protein or peptide as biomarker, preferably refers to a binding reaction wherein a binding agent binds to the corresponding biomarker with an affinity (“association constant” Kₐ) of at least 10⁷ M⁻¹. The term “specific binding” or “specifically binds” preferably refers to an affinity of at least 10⁸ M⁻¹ or even more preferred of at least 10⁹ M⁻¹ for its target molecule. The term “specific” or “specifically” is used to indicate that other molecules present in the sample do not significantly bind to the binding agent specific for the target molecule.

In one embodiment, the method of the present invention is based on detecting a protein complex comprising human CES-2 and a non-human or chimeric CES-2-specific binding agent. In such embodiment the present invention reads on a method for assessing atrial fibrillation in a subject, said method comprising the steps of (a) incubating a sample from said subject with a non-human CES-2-specific binding agent (b) measuring the complex between the CES-2-specific binding agent and CES-2 formed in (a), and (c) comparing the measured amount complex to a reference amount. An amount of the complex at or above the reference amount is indicative for the diagnosis (and thus the presence) of atrial fibrillation, the presence of persistent atrial fibrillation, a subject who shall be subjected to ECG, or a subject who is at risk of an adverse event. An amount of the complex below the reference amount is indicative for the absence of atrial fibrillation; the presence of paroxysmal atrial fibrillation, a subject who is shall be not subjected to ECG, or a subject who is not at risk of an adverse event.

The term “amount” as used herein encompasses the absolute amount of a biomarker as referred to herein (such as CES-2 or the natriuretic peptide), the relative amount or concentration of the said biomarker as well as any value or parameter which correlates thereto or can be derived therefrom. Such values or parameters comprise intensity signal values from all specific physical or chemical properties obtained from the said peptides by direct measurements, e.g., intensity values in mass spectra or NMR spectra. Moreover, encompassed are all values or parameters which are obtained by indirect measurements specified elsewhere in this description, e.g., response amounts determined from biological read out systems in response to the peptides or intensity signals obtained from specifically bound ligands. It is to be understood that values correlating to the aforementioned amounts or parameters can also be obtained by all standard mathematical operations.

The term “comparing” as used herein refers to comparing the amount of the biomarker (such as CES-2 and the natriuretic peptide such as NT-proBNP or BNP and/or ESM-1, ANG-2, IGFBP7) in the sample from the subject with the reference amount of the biomarker specified elsewhere in this description. It is to be understood that comparing as used herein usually refers to a comparison of corresponding parameters or values, e.g., an absolute amount is compared to an absolute reference amount while a concentration is compared to a reference concentration or an intensity signal obtained from the biomarker in a sample is compared to the same type of intensity signal obtained from a reference sample. The comparison may be carried out manually or computer-assisted. Thus, the comparison may be carried out by a computing device. The value of the determined or detected amount of the biomarker in the sample from the subject and the reference amount can be, e.g., compared to each other and the said comparison can be automatically carried out by a computer program executing an algorithm for the comparison. The computer program carrying out the said evaluation will provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provide the desired assessment in a suitable output format. For a computer-assisted comparison, the value of the determined amount may be compared to values corresponding to suitable references which are stored in a database by a computer program. The computer program may further evaluate the result of the comparison, i.e. automatically provides the desired assessment in a suitable output format.

In accordance with the present invention, the amount of the biomarker CES-2 and optionally the amount of the natriuretic peptide and/or of natriuretic peptide, ESM-1, ANG-2, IGFBP7 shall be compared to a reference. The reference is preferably a reference amount. The term “reference amount” is well understood by the skilled person. It is to be understood that the reference amount shall allow for the herein described assessment of atrial fibrillation. E.g., in connection with the method for diagnosing atrial fibrillation, the reference amount preferably refers to an amount which allows for allocation of a subject into either (i) the group of subjects suffering from atrial fibrillation or (ii) the group of subjects not suffering from atrial fibrillation. A suitable reference amount may be determined from a reference sample to be analyzed together, i.e. simultaneously or subsequently, with the test sample.

It is to be understood that the amount of CES-2 is compared to a reference amount for a natriuretic peptide, whereas the amount of the natriuretic peptide is compared to a reference amount of the natriuretic peptide. If the amounts of two markers are determined, it is also envisaged that a combined score is calculated based on the amounts of CES-2 and the natriuretic peptide. In a subsequent step, the score is compared to a reference score.

Furthermore, it is to be understood that the amount of CES-2 is compared to a reference amount for ESM1, whereas the amount of the ESM1 is compared to a reference amount of the ESM1. If the amounts of two markers are determined, it is also envisaged that a combined score is calculated based on the amounts of CES-2 and the ESM1. In a subsequent step, the score is compared to a reference score.

Furthermore, it is to be understood that the amount of CES-2 is compared to a reference amount for ANG-2, whereas the amount of the ANG-2 is compared to a reference amount of the ANG-2. If the amounts of two markers are determined, it is also envisaged that a combined score is calculated based on the amounts of CES-2 and the ANG-2. In a subsequent step, the score is compared to a reference score.

Furthermore, it is to be understood that the amount of CES-2 is compared to a reference amount for IGFBP7, whereas the amount of the IGFBP7 is compared to a reference amount of the IGFBP7. If the amounts of two markers are determined, it is also envisaged that a combined score is calculated based on the amounts of CES-2 and the IGFBP7. In a subsequent step, the score is compared to a reference score.

Reference amounts can, in principle, be calculated for a cohort of subjects as specified above based on the average or mean values for a given biomarker by applying standard methods of statistics. In particular, accuracy of a test such as a method aiming to diagnose an event, or not, is best described by its receiver-operating characteristics (ROC) (see especially Zweig M H. et al., Clin. Chem. 1993; 39:561-577). The ROC graph is a plot of all the sensitivity versus specificity pairs resulting from continuously varying the decision threshold over the entire range of data observed. The clinical performance of a diagnostic method depends on its accuracy, i.e. its ability to correctly allocate subjects to a certain prognosis or diagnosis. The ROC plot indicates the overlap between the two distributions by plotting the sensitivity versus 1—specificity for the complete range of thresholds suitable for making a distinction. On the y-axis is sensitivity, or the true-positive fraction, which is defined as the ratio of number of true-positive test results to the product of number of true-positive and number of false-negative test results. It is calculated solely from the affected subgroup. On the x-axis is the false-positive fraction, or 1—specificity, which is defined as the ratio of number of false-positive results to the product of number of true-negative and number of false-positive results. It is an index of specificity and is calculated entirely from the unaffected subgroup. Because the true- and false-positive fractions are calculated entirely separately, by using the test results from two different subgroups, the ROC plot is independent of the prevalence of the event in the cohort. Each point on the ROC plot represents a sensitivity/1—specificity pair corresponding to a particular decision threshold. A test with perfect discrimination (no overlap in the two distributions of results) has an ROC plot that passes through the upper left corner, where the true-positive fraction is 1.0, or 100% (perfect sensitivity), and the false-positive fraction is 0 (perfect specificity). The theoretical plot for a test with no discrimination (identical distributions of results for the two groups) is a 45° diagonal line from the lower left corner to the upper right corner. Most plots fall in between these two extremes. If the ROC plot falls completely below the 45° diagonal, this is easily remedied by reversing the criterion for “positivity” from “greater than” to “less than” or vice versa. Qualitatively, the closer the plot is to the upper left corner, the higher the overall accuracy of the test. Dependent on a desired confidence interval, a threshold can be derived from the ROC curve allowing for the diagnosis for a given event with a proper balance of sensitivity and specificity, respectively. Accordingly, the reference to be used for the method of the present invention, i.e. a threshold which allows assessing atrial fibrillation can be generated, preferably, by establishing a ROC for said cohort as described above and deriving a threshold amount therefrom. Dependent on a desired sensitivity and specificity for a diagnostic method, the ROC plot allows deriving a suitable threshold. It will be understood that an optimal sensitivity is desired for e.g. excluding a subject being at risk of stroke (i.e. a rule out) whereas an optimal specificity is envisaged for a subject to be predicted to be at risk of stroke (i.e. a rule in).

Preferably, the term “reference amount” herein refers to a predetermined value. Said predetermined value shall allow for predicting the risk of stroke.

Preferably, the reference amount, i.e. the reference amount shall allow for differentiating between a subject who is at risk of suffering from stroke and a subject who is not at risk of suffering from stroke.

The diagnostic algorithm is preferably as follows:

Preferably, an amount of CES-2 which is decreased and the amounts one or more biomarkers comprising of a natriuretic peptide, ESM-1, ANG-2, IGFBP7 which are increased as compared to the reference amount is indicative for a subject who is at risk to suffer from stroke.

Preferably, an amount of CES-2 which is increased or not altered and the amounts one or more biomarkers comprising of a natriuretic peptide, ESM-1, ANG-2, IGFBP7 which are decreased or not altered as compared to the reference amount is indicative for a subject who is not at risk to suffer from stroke.

Preferred reference amounts are given in the Examples section. However, it will be understood by the skilled person that depending on the desired sensitivity and specificity other reference amounts would also allow for a reliable prediction.

In the studies underlying the present invention, it has been further shown that the determination of the amount of CES-2 and one or more biomarkers comprising of a natriuretic peptide, ESM-1, ANG-2, IGFBP7 allow for improving the prediction accuracy of a clinical stroke risk score for a subject. Thus, the combined determination of clinical stroke risk score and the determination of the amount of CES-2 and one or more biomarkers comprising of a natriuretic peptide, ESM-1, ANG-2, IGFBP7 allows for an even more reliable prediction of stroke as compared to the determination of CES-2 and the determination of the clinical stroke risk score alone.

Accordingly, the method for predicting the risk of stroke may further comprise the combination of the amount of CES-2 and one or more biomarkers comprising of a natriuretic peptide, ESM-1, ANG-2, IGFBP7 with the clinical stroke risk score. Based on the combination of the amount of CES-2 and one or more biomarkers comprising of a natriuretic peptide, ESM-1, ANG-2, IGFBP7 and the clinical risk score, the risk of stroke of the test subject is predicted.

Accordingly, the present invention in particular relates to a method for predicting the risk of stroke in a subject, comprising the steps of
a) determining the amount of CES-2 and/or the amount of one or more biomarkers comprising of a natriuretic peptide, ESM-1, ANG-2, IGFBP7 in a sample from the subject having a known clinical stroke risk score, and
b) assessing the clinical stroke risk score for said subject, and
c) predicting the risk of stroke based on the results of steps a) and b).

In accordance with the method of the present invention, it is envisaged that the subject is a subject who has a known clinical stroke risk score. Accordingly, the value for the clinical stroke risk score is known for the subject.

Alternatively, the method may comprise obtaining or providing the value for the clinical stroke risk score. Accordingly, step b) preferably comprises providing the value for the clinical risk score. Preferably, the value is a number. In an embodiment, the clinical stroke risk score is generated by one of the clinically based tools available to physicians. Preferably, the value provided by determining the value for the clinical stroke risk score for the subject. More preferably, the value for the subject is obtained from patient record databases and medical history of the subject. The value for the score therefore can be also determined using historical or published data of the subject.

In accordance with the present invention, the amount of ANG-2 and/or IGFBP7 is combined with the clinical stroke risk score. This means preferably that a value for the amount of ANG-2 and/or IGFBP7 is combined with the clinical stroke risk score. Accordingly, the values are operatively combined to predict the risk of the subject to suffer from stroke. By combining the value, a single value may be calculated, which itself can be used for the prediction.

Clinical stroke risk scores are well known in the art. E.g. said scores are described in Kirchhof P. et al., (European Heart Journal 2016; 37: 2893-2962). In an embodiment, the score is CHA₂DS₂-VASc-Score. In another embodiment, the score is the CHADS₂ Score. (Gage B F. Et al., JAMA, 285 (22) (2001), pp. 2864-2870) and ABC score, i.e. the ABC (age, biomarkers, clinical history) stroke risk score (Hijazi Z. et al., Lancet 2016; 387(10035): 2302-2311). All publications in this paragraph are herewith incorporated by reference with respect to their entire disclosure content.

Thus, in an embodiment of the present invention, the clinical stroke risk score is the CHA₂DS₂-VASc-Score.

In another embodiment of the present invention, the clinical stroke risk score is the CHADS₂ Score.

In a further embodiment, the clinical risk score is the ABC Score. The ABC stroke risk score is a novel biomarker-based risk score for predicting stroke in AF was validated in a large cohort of patients with AF and further externally validated in an independent AF cohort (see Hijazi et al., 2016). It includes the age of the subject, the blood, serum or plasma levels of cardiac Troponin T and NT-proBNP in said subject, and information on whether the subject has a history of stroke. Preferably, the ABC stroke score is the score as disclosed in Hijazi et al.

In a preferred embodiment, the above method for predicting the risk of stroke in a subject further comprises the step of recommending anticoagulation therapy or of recommending an intensification of anticoagulation therapy if the subject has been identified to be at risk to suffer from stroke (as described elsewhere herein).

### Method for Improving the Prediction Accuracy of a Clinical Stroke Risk Score

The present invention further relates to a method for improving the prediction accuracy of a clinical stroke risk score for a subject, comprising the steps of
a) determining the amount of CES-2 and/or the amount of one or more biomarkers comprising of a natriuretic peptide, ESM-1, ANG-2, IGFBP7, and
b) combining a value for the amount of CES-2 and/or the amount of one or more biomarkers comprising of a natriuretic peptide, ESM-1, ANG-2, IGFBP7 with the clinical stroke risk score, whereby the prediction accuracy of said clinical stroke risk score is improved.

The method may comprise the further step of c) improving prediction accuracy of said clinical stroke risk score based on the results of step b).

The definitions and explanations given herein above in connection with the method of assessing atrial fibrillation, in particular of predicting the risk of an adverse event (such as stroke) preferably apply to the aforementioned method as well E.g., it envisaged that the subject is a subject who has a known clinical stroke risk score. Alternatively, the method may comprise obtaining or providing the value for the clinical stroke risk score.

In accordance with the present invention, the amount of CES-2 and/or the amount of one or more biomarkers comprising of a natriuretic peptide, ESM-1, ANG-2, IGFBP7 is combined with the clinical stroke risk score. This means preferably, that the value for the amount of CES-2 and/or a natriuretic peptide and/or ESM-1 and/or ANG-2 and/or IGFBP7 is combined with the clinical stroke risk score. Accordingly, the values are operatively combined to improve the prediction accuracy of said clinical stroke risk score.

The present invention further concerns a method of aiding in the prediction of the risk of stroke of a subject, said method comprising the steps of:
a) obtaining a sample from a subject as referred to herein in connection with the method of the present invention,
b) determining the amount of CES-2 and optionally one or more biomarkers comprising of a natriuretic peptide, ESM-1, ANG-2, IGFBP7 in a sample from the subject, and
c) providing information on the determined amount of the CES-2 and optionally one or more biomarkers comprising of a natriuretic peptide, ESM-1, ANG-2, IGFBP7 to the attending physician of the subject, thereby aiding in the prediction of the risk.

Step a) of the aforementioned method of obtaining the sample does not encompass the draw-ing of the sample from the subject. Preferably, the sample is obtained by receiving a sample from said subject. Thus, the sample can have been delivered.

Method for Diagnosing Atrial Fibrillation

The term “diagnosing” as used herein means assessing whether a subject as referred to in accordance with the method of the present invention suffers from atrial fibrillation (AF), or not. In an embodiment, it is diagnosed that a subject suffers from AF. In a preferred embodiment, it is diagnosed that a subject suffers from paroxysmal AF. In an alternative embodiment, it is diagnosed that a subject does not suffer from AF.

In accordance with the present invention, all types of AF can be diagnosed. Thus, the atrial fibrillation may be paroxysmal, persistent or permanent AF. Preferably, the parxoxysmal or atrial fibrillation are diagnosed, in particular in a subject not suffering from permanent AF.

The actual diagnosis whether a subject suffers from AF, or not may comprise further steps such as the confirmation of a diagnosis (e.g. by ECG such as Holter-ECG). Thus, the present invention allows for assessing the likelihood that a patient suffers from atrial fibrillation. A subject who has an amount of CES-2 above the reference amount is likely to suffer from atrial fibrillation, whereas a subject who has an amount of CES-2 below the reference amount is not likely to suffer from atrial fibrillation. Accordingly, the term “diagnosing” in the context of the present invention also encompasses aiding the physician to assess whether a subject suffers from atrial fibrillation, or not.

Preferably, an amount of CES-2 (and optionally an amount of one or more biomarkers comprising of a natriuretic peptide, ESM-1, ANG-2, IGFBP7) in the sample from a test subject which is (are) increased as compared to the reference amount (or to the reference amounts) is indicative for a subject suffering from atrial fibrillation, and/or an amount of CES-2 (and optionally an amount of one or more biomarkers comprising of a natriuretic peptide, ESM-1, ANG-2, IGFBP7) in the sample from a subject which is (are) decreased as compared to the reference amount (or the reference amounts) is indicative for a subject not suffering from atrial fibrillation.

In a preferred embodiment, the reference amount, i.e. the reference amount CES-2 and, if a natriuretic peptide is determined, the reference amount for the natriuretic peptide, shall allow for differentiating between a subject suffering from atrial fibrillation and a subject not suffering from atrial fibrillation. Preferably, said reference amount is a predetermined value.

In a further preferred embodiment, the reference amount, i.e. the reference amount for CES-2 and, if a natriuretic peptide, ESM-1, ANG-2, IGFBP7 are determined, the reference amounts for a natriuretic peptide, ESM-1, ANG-2, IGFBP7, shall allow for differentiating between a subject suffering from atrial fibrillation and a subject not suffering from atrial fibrillation. Preferably, said reference amount (s) is (are) a predetermined value(s).

In an embodiment, the method of the present invention allows for the diagnosis of a subject suffering from atrial fibrillation. Preferably, the subject is suffering from AF, if the amount of CES-2 (and optionally the amounts of the natriuretic peptide, ESM-1, ANG-2, IGFBP7) is (are) above the reference amount. In an embodiment, the subject is suffering from AF, if the amount of CES-2 is above a certain percentile (e.g. 99^{th} percentile) upper reference limit (URL) of a reference amount.

In another preferred embodiment, the method of the present invention allows for the diagnosis that a subject is not suffering from atrial fibrillation. Preferably, the subject is not suffering from AF, if the amount of CES-2 (and optionally the amounts of the natriuretic peptide, ESM-1, ANG-2, IGFBP7) is (are) below the reference amount (such as the certain percentile URL). Thus, in an embodiment, the term “diagnosing atrial fibrillation” refers to “ruling out atrial fibrillation”.

Ruling-out out atrial fibrillation is of particular interest since further diagnostic tests for the diagnosis of atrial fibrillation such as an ECG test can be avoided. Thus, thanks to the present invention, unnecessary health care costs can be avoided.

Accordingly, the present invention also concerns a method for ruling out atrial fibrillation, comprising the steps of
a) determining the amount of CES-2 in a sample from the subject, and
b) comparing the amount of CES-2 to a reference amount whereby atrial fibrillation is ruled out.

Preferably, an amount of the biomarker CES-2 in the sample of the subject which is decreased as compared to the reference amount (such as a reference for ruling out atrial fibrillation) is indicative for a subject who does not suffer from atrial fibrillation, and thus for ruling out atrial fibrillation in the subject. E.g. the reference amount for CES-2 may be determined in a sample from a subject who does not suffer from AF, or in samples of a group thereof.

When determining the biomarker CES-2 and the biomarkers a natriuretic peptide, ESM-1, ANG-2, IGFBP7 in combination, an even more reliable rule-out can be achieved. Accordingly, steps a) and b) are preferably as follows:
a) determining the amount of CES-2 and the amount of one or more biomarkers comprising of a natriuretic peptide, ESM-1, ANG-2, IGFBP7 in a sample from the subject, and
b) comparing the amount of CES-2 and the amount of one or more biomarkers comprising of a natriuretic peptide, ESM-1, ANG-2, IGFBP7 to reference amounts whereby atrial fibrillation is ruled out.

Preferably, amounts of both biomarkers, i.e. the amount of the biomarker CES-2 and the amount of the natriuretic peptide,

or the amounts of both biomarkers, i.e. the amount of the biomarker CES-2 and the amount of ESM1,

or the amounts of three biomarkers, i.e. the amount of the biomarker CES-2, the amount of the natriuretic peptide and the amount of ESM1,

in the sample of the subject which are decreased as compared to the respective reference amount (such as a reference amount for ruling out atrial fibrillation) are indicative for a subject who does not suffer from atrial fibrillation, and thus for ruling out atrial fibrillation in the subject. E.g. the reference amount for the natriuretic peptide and/or ESM1 may be determined in a sample from a subject who does not suffer from AF, or in samples of a group thereof.

In an embodiment of the method of diagnosing atrial fibrillation, said method further comprises a step of recommending and/or initiating a therapy for atrial fibrillation based on the results of the diagnosis. Preferably, a therapy is recommended or initiated if it is diagnosed that the subject suffers from AF. Preferred therapies for atrial fibrillation are disclosed elsewhere herein.

The present invention further relates to a method, comprising:
a) providing a test for the biomarker CES-2 and optionally one or more biomarkers comprising of a natriuretic peptide, ESM-1, ANG-2, IGFBP7, and
b) providing instructions for using of test results obtained or obtainable by said test(s) in the assessment of atrial fibrillation.

The purpose of the aforementioned method is, preferably, the aid in the prediction of the risk of stroke as described elsewhere herein in more detail.

The instructions shall contain a protocol for carrying out the method of assessing atrial fibrillation as described herein above. Further, the instructions shall contain at least one value for a reference amount for CES-2 and/or for a natriuretic peptide and/or ESM-1 and/or ANG-2 and/or IGFBP7.

The “test” is preferably a kit adapted to carry out the method of assessing atrial fibrillation. The term “Kit” is explained herein below. E.g. said kit shall comprise at least one detection agent for the biomarker ANG-2 and/or at least one detection agent for the biomarker IGFBP7. The detection agents for the two biomarkers can be provided in a single kit or in two separate kits.

The test result obtained or obtainable by said test, is the value for the amount of the biomarker(s).

In an embodiment, step b) comprises providing instructions for using of test results obtained or obtainable by said test(s) in prediction of stroke (as described herein elsewhere).

The definitions and explanations given herein above, preferably, apply mutatis mutandis to the following:

The present invention further relates to the use of
i) the biomarker CES-2 and and optionally of one or more biomarkers comprising of a natriuretic peptide, ESM-1, ANG-2, IGFBP7
ii) at least one detection agent that specifically binds to CES-2, and optionally at least one detection agent that specifically binds to one or more biomarkers comprising of a natriuretic peptide, ESM-1, ANG-2, IGFBP7 in a sample from a subject for a) assessing the risk of stroke or b) for assessing the efficacy of an anticoagulation therapy or c)

The present invention further contemplates to the use of
i) the biomarker CES-2 and/or
ii) at least one detection agent that specifically binds to CES-2,
   in a sample from a subject,
   in combination with a clinical stroke risk score,
   for predicting the risk of a subject to suffer from stroke.

Finally, the present invention further relates to the use of
i) the biomarker CES-2 and/or
ii) at least one detection agent that specifically binds to CES-2 in a sample from a subject for predicting the efficacy of an anticoagulation therapy of a subject.

The terms mentioned in connection with the aforementioned use such as “sample”, “subject”, “detection agent”, “CES-2”, “natriuretic peptide”, “ESM-1”, “ANG-2”, “IGFBP7”, “specifically binding”, “stroke”, and “prediction the risk” have been defined in connection with the methods of the present invention. The definitions and explanations apply accordingly.

Preferably, the aforementioned uses are in vitro uses. Moreover, the detection agent is, preferably, an antibody such as a monoclonal antibody (or an antigen binding fragment thereof) which specifically binds to the biomarker.

All references cited in this specification are herewith incorporated by reference with respect to their entire disclosure content and the disclosure content specifically mentioned in this specification.

The figures show:

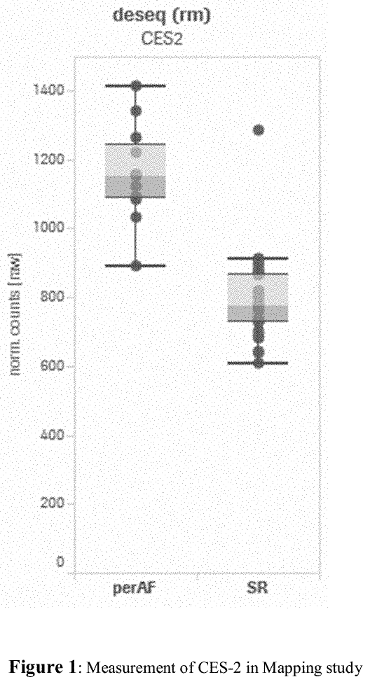

FIG. 1: Measurement of CES-2 in Mapping study: Exploratory AFib panel: Patients with a history of atrial fibrillation undergoing open chest surgery and epicardial mapping of paroxysmal AF, persistent AF or SR (Mapping study). Atrial tissue RNA expression profiles were assessed.

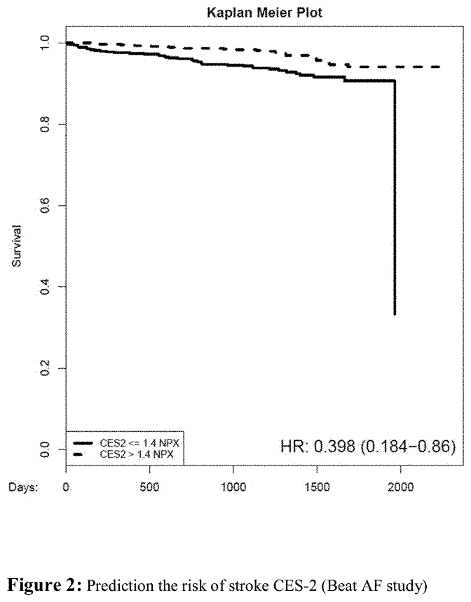

FIG. 2: Prediction the risk of stroke CES-2 vs parameters of clinical risk scores (Beat AF study: The Figure shows, that reduced titers of CES-2 associate to increased risk of stroke. CES-2 improved the C-Index of several clinical risk scores.

FIG. 3: Correlation to NTproBNP and ESM-1 in Beat AF: FIG. 3 shows that CES-2 has almost no correlation with established markers (NTproBNP and ChadsVasc) as well as with ESM1.

FIG. 4: CES-2 values observed in the BEAT-AF study separated by intake of oral anticoagulation: Patients which use Rivaroxaban show higher concentrations of CES-2 compared to the remaining patients.

a) CES-2 vs NTproBNP correlation coefficient=−0.19
b) CES-2 vs ESM1 correlation coefficient=−0.18
c) CES-2 vs CHADsVASc correlation coefficient=−0.12

These data suggest, that CES-2 provides complementary information and combinations of CES-2 and/or NTproBNP and/or ESM1 and/or ANG-2 and/or IGFBP7 and/or CHADsVASc markers may provide improved detection of patients at high risk of stroke versus each marker alone. These data further suggest that CES-2 can be used to diagnose the disease, to classify the disease, to assess the disease severity, to guide therapy (with objectives to therapy intensification/reduction), to predict disease outcome (risk prediction, e.g. stroke), therapy monitoring (e.g., effect of anti-angionetic drugs on CES-2 levels), therapy stratification (selection of therapy options; e.g. long-term from Beat AF and selection)

### EXAMPLES

The invention will be merely illustrated by the following Examples. The said Examples shall, whatsoever, not be construed in a manner limiting the scope of the invention.

### Example 1: Differential Expression of CES-2 in Cardiac Tissue of AF Patients

Differential CES-2 expression levels have been determined in myocardial tissue samples from the right atrial appendage of n=40 patients.

RNAseq analyses

Atrial tissue was sampled during open chest surgery because of CABG or valve surgery. Evidence of AF or SR (controls) was generated during surgery with simultaneous Endo-Epicardial High Density Activation Mapping. Patients with AF and controls were matched with regard to gender, age and comorbidities.

Atrial tissue samples were prepared for
AF patients; n=11 patients
control patients in SR; n=39 patients.

Differential expression of CES-2 was determined in RNAseq analyses applying the algorithms RSEM and DESEQ2.

As shown in FIG. 2, CES-2 expression was found to be upregulated in the analyzed atrial tissues of the 11 patients with persistent AF versus the 29 control patients.

The fold change in expression (FC) was 1,439 The FDR (false discovery rate) was 0,00000000036.

The altered expression of CES-2 was determined in the damaged end organ, the atrial tissue. CES-2 mRNA levels were compared to results of high density mapping of the atrial tissue. Elevated CES-2 mRNA levels were detected in atrial tissue samples with conduction disturbances as characterized by electrical mapping. Conductance disturbances may be caused by fat infiltration or by interstitial fibrosis. The observed differential expression of CES-2 in atrial tissue of patients suffering from atrial fibrillation supports, that CES-2 is released in the circulation from the myocardium, in particular from the right atrial appendage and elevated serum/plasma titers assist the detection of episodes of AF.

It is concluded, that CES-2 is released from the heart into the blood and may aid the detection of AF episodes.

### Example 2: Prediction of Stroke

Analysis Approach

The ability of circulating CES-2 to predict the risk for the occurrence of stroke was assessed in a prospective, multicentric registry of patients with documented atrial fibrillation (Conen D., Forum Med Suisse 2012; 12:860-862). CES-2 was measured using a stratified case cohort design as described in Borgan (2000).

For each of the 70 patients which experienced a stroke during follow up (“events”), 1 matched control was selected. Controls were matched based on the demographic and clinical information of age, sex, history of hypertension, atrial fibrillation type and history of heart failure (CHF history).

CES-2 results were available for 69 patients with an event and 69 patients without an event.

CES-2 was measured using the Olink platform therefor no absolute concentration values are available and can be reported. Results will be reported on an arbitrary signal scale (NPX).

In order to quantify the univariate prognostic value of CES-2 proportional hazard models were used with the outcome stroke.

The univariate prognostic performance of CES-2 was assessed by two different incorporations of the prognostic information given by CES-2.

The first proportional hazard model included CES-2 binarized at the median (1.4 NPX) and therefore comparing the risk of patients with CES-2 below or equal to the median versus patient with CES-2 above the median.

The second proportional hazard model included the original CES-2 levels but transformed to a log 2 scale. The log 2 transformation was performed in order to enable a better model calibration.

Because the estimates from a naïve proportional hazard model on the case control cohort would be biased (due to the altered proportion of cases to controls) a weighted proportional hazard model was used. Weights are based on the inverse probability for each patient to be selected for the case control cohort as described in Mark (2006).

In order to get estimates for the absolute survival rates in the two groups based on the dichotomized baseline CES-2 measurement (<=1.4 NPX vs >1.4 NPX) a weighted version of the Kaplan-Meier plot was created as described in Mark (2006).

In order to assess if the prognostic value of CES-2 is independent from known clinical and demographic risk factors a weighted proportional cox model including in addition the variables age, sex, CHF history, history of hypertension, Stroke/TIA/Thromboembolism history, vascular disease history and diabetes history was calculated.

In order to assess the ability of CES-2 to improve existing risk scores for the prognosis of stroke the CHADS₂ the CHA₂DS₂-VASc and the ABC score were extended by CES-2 (log 2 transformed). Extension was done by creating a portioned hazard model including CES-2 and the respective risk score as independent variables.

The c-indices of the CHADS₂, the CHA₂DS₂-VASc and ABC score were compared to the c-indices of these extended models. For the calculation of the c-index in the case-cohort setting a weighted version of the c-index was used as proposed in Ganna (2011).

Results

Table 1 shows the results of the two univariate weighted proportional hazard models including the binarized or the log 2 transformed CES-2.

The association between the risk for experiencing a stroke with the baseline value of CES-2 is significant in both models.

The hazard ration for the binarized CES-2 implies a 0.4-fold lower risk for a stroke in the patient group with baseline CES-2>1.4 NPX versus the patient group with baseline CES-2<=1.4 NPX. The results of the proportional hazard model including CES-2 as log 2 transformed linear risk predictor suggest the log 2 transformed values CES-2 are negatively correlated to the risk for experiencing a stroke. The hazard ratio of 0.14 can be interpreted in a way that a 2-fold increase of CES-2 is associated with 0.14 decrease of risk for a stroke. In this context it is interesting to note that CES-2 level correlate with the intake of certain oral anticoagulants (OAKs). FIG. 4 shows that patients which use Rivaroxaban show higher concentrations of CES-2 compared to the remaining patients. But there are also some patients with intake of Rivaroxaban which have CES values below 1.4 NPX. This could indicate that CES-2 could be used to monitor the effectiveness of OAK intake.

TABLE 1
Results result of the univariate weighted proportional hazard
model including the binarized and log2 transformed CES-2.

| | Hazard Ratio | | |
|---|---|---|---|
| | (HR) | 95%-CI HR | P-Value |
| CES-2 log2 | 0.138 | 0.0235-0.8055 | 0.028 |
| Baseline CES-2 > 1.4 | 0.4116 | 0.1966-0.8618 | 0.019 |
| NPX vs CES-2 <= | | | |
| 1.4 NPX | | | |

Table 2 shows the results of a proportional hazard model including CES-2 (log 2 transformed) in the combination with clinical and demographic variables.

The effect of CES-2 remains significant and the HR is now 0.09 for the lo2 transformed CES-2.

TABLE 2
Multivariate proportional hazard model including CES-2
and relevant clinical and demographic variables.

| | Hazard Ratio | | |
|---|---|---|---|
| | (HR) | 95%-CI HR | P-Value |
| History hypertension | 1.7327 | 0.655-4.5835 | 0.2681 |
| Age | 1.0225 | 0.9791-1.0679 | 0.3145 |
| History | 1.8311 | 0.7158-4.6843 | 0.2069 |
| Stroke/TIA/embolism | | | |
| Sex = male | 0.5124 | 0.2218-1.1837 | 0.1175 |
| History CHF | 0.7825 | 0.3404-1.7984 | 0.5634 |
| History vascular | 1.1212 | 0.4705-2.6718 | 0.7962 |
| disease | | | |
| CES-2 (log2 | 0.0947 | 0.0144-0.6237 | 0.0142 |
| transformed) | | | |

Table 3 shows the results of the weighted proportional hazard model combining the CHADS₂ score with CES-2 (log 2 transformed). Also in this model CES-2 can add prognostic information to the CHADS₂ score.

TABLE 3
Weighted proportional hazard model combining the
CHADS₂ score with CES-2 (log2 transformed)

| | Hazard Ratio | | |
|---|---|---|---|
| | (HR) | 95%-CI HR | P-Value |
| CHADS₂ score | 1.3892 | 1.0733-1.7980 | 0.0125 |
| CES-2 (log2 | 0.1271 | 0.0203-0.7964 | 0.0276 |
| transformed) | | | |

Table 4 shows the results of the weighted proportional hazard model combining the CHA₂DS₂-VASc score with CES-2 (log 2 transformed). Again CES-2 adds prognostic information.

TABLE 4
Weighted proportional hazard model combining the
CHA₂DS₂-VASc score with CES-2 (log2 transformed)

| | Hazard Ratio | | |
|---|---|---|---|
| | (HR) | 95%-CI HR | P-Value |
| CHA₂DS₂-VASc | 1.3862 | 1.1191-1.7172 | 0.0028 |
| score | | | |
| CES-2 (log2 | 0.1113 | 0.0180-0.6874 | 0.0181 |
| transformed) | | | |

Table 5 shows the results of the weighted proportional hazard model combining the ABC score with CES-2 (log 2 transformed). The prognostic additional value of CES-2 decreases slightly but stays significant.

TABLE 5
Weighted proportional hazard model combining
the ABC score with CES-2 (log2 transformed)

| | Hazard Ratio | | |
|---|---|---|---|
| | (HR) | 95%-CI HR | P-Value |
| ABC score | 1.1289 | 1.0171-1.2530 | 0.0227 |
| CES-2 (log2 | 0.1804 | 0.0338-0.9613 | 0.0448 |
| transformed) | | | |

Table 6 shows the estimated c-indexes of CES-2 alone, of the CHADS₂, the CHA₂DS₂-VASc and the ABC score and of the weighted proportional hazard model combining the CHADS₂, the CHA₂DS₂-VASc and the ABC score with CES-2 (log 2).

The addition of CES-2 to CHA₂DS₂-VASc score improves the c-index by 0.0611 which can be considered as a clinical meaningful improvement of the risk prediction.

For the CHADS₂ score the c-index improvement is comparable with 0.0646 as for the ABC score with 0.0617.

TABLE 6
C-indexes of CES-2, the CHADS₂, CHA₂DS₂-VASc
and ABC score and their combination with CES-2.

| | C-Index |
|---|---|
| CES-2 univariate | 0.7080 |
| CHADS₂ | 0.6505 |
| CHADS₂ + CES-2 | 0.7151 |
| CHA₂DS₂-VASc | 0.6740 |
| CHA₂DS₂-VASc + CES-2 | 0.7350 |
| ABC score | 0.6484 |
| ABC score + CES-2 | 0.7101 |

### Example 3: Biomarker Measurements

CES-2 was measured in a commercially available O-link multi-marker panel for (Carboxy-lesterase-2 (CES-2); Proximity Extension Assay from O-link, Sweden.

Case Studies

The CHA2DS2-VASc score predicts incidence of stroke in patients with and also without atrial fibrillation (https://www.ncbi.nlm nih.gov/pubmed/29754652); however, it is less clear, if and at what CHA2DS2-VASc score the patients without atrial fibrillation should receive oral anticoagulation (OAC) and at which dose, so that biomarkers such as CES-2 help to assess the need for therapy and effectiveness of OAC.

A 70-year-old male patient with hypertension and no history of atrial fibrillation presents in sinus rhythm. CES2 is determined in an EDTA plasma sample obtained from the patient. The CES2 value is below a reference value. The reduced CES2 titers in combination of other stroke risk parameters (advanced age and hypertension) are indicative of high risk to experience a stroke. As consequence the patient is admitted to an anticoagulation therapy.

A 75-year-old female patient without a history of atrial fibrillation requests a checkup at the doctor's office. The patient presents in sinus rhythm, however structural heart disease is diagnosed. The patient already receives direct oral anticoagulation therapy (at low starting dose) because of a history of stroke and high overall CHA2DS2-VASc score. In order to determine the current risk of stroke, CES2 is measured in a serum sample obtained from the patient. The observed CES2 value is below a reference value. The reduced CES2 titers in combination of other risk parameters (history of stroke) are indicative of a high risk of stroke. As consequence the dosage of the anticoagulation therapy is increased.

A 68-year-old obese female patient with Diabetes Mellitus and heart failure with reduced ejection fraction presents with acute symptoms of shortness of breath. In prior visits, he patient has no history of atrial fibrillation. According to a high overall CHA2DS2-VASC risk score, the physician decided to start oral anticoagulation (low dose) even in the absence of AFib. The CES-2 level was determined before and after onset of anticoagulation. The patient now is wondering whether the anticoagulation therapy is effective and still necessary. In order to specify the acute risk of stroke CES2 is determined in a EDTA sample obtained from the patient. The observed CES2 value is above a reference value. The increased CES2 titers are indicative of an effective anticoagulation therapy. As consequence the anticoagulation therapy is maintained.
Related, very recent research question “Does CHA2DS2VASc score predict incidence of stroke in patients without A-Fib/Flutter?” or “how much risk points does AFib adds to the CHA₂DS₂-VASc (eg, a 7-fold risk, but how many points]” and first results from 2014-2019:
   “The event rates were 0.67%/y for ischemic stroke or MI, 0.96%/y for AF, and 0.52%/y for major bleeding “https://www.ncbi.nlm.nih.gov/pubmed/29754652 (related also: Circulation. 2017; 136:A20985)
   “In patients with ACS but no AF, the CHADS2 and CHA2DS2-VASc scores predict ischaemic stroke/TIA events with similar accuracy to that observed in historical populations with non-valvular AF, but with lower absolute event rates.” https://www.ncbi.nlm nih.gov/pubmed/24860007
   “The CHA2DS2-VASc tool predicts thromboembolic events and overall mortality in patients without atrial fibrillation who have implantable devices” https://www.ncbi.nlm nih.gov/pubmed/28259228
   “The absolute risk of thromboembolic complications was higher among patients without AF compared with patients with concomitant AF at high CHA2DS2-VASc scores.” https://www.ncbi.nlm nih.gov/pubmed/26318604.

1. A method for predicting the risk of stroke in a subject, comprising the steps of
a) determining the amount of CES-2 and optionally one or more biomarkers selected from the group consisting of a natriuretic peptide, ESM-1, ANG-2, and IGFBP7 in a sample from the subject, and
b) comparing the amount of CES-2 and optionally one or more biomarkers selected from the group consisting of a natriuretic peptide, ESM-1, ANG-2, and IGFBP7, to a reference amount (or to reference amounts), whereby the risk of stroke is to be predicted.

2. The method of claim 1, wherein the amount of CES-2 in the sample from the subject is decreased as compared to the reference amount (or to reference amounts).

3. The method according to claim 1, wherein further comprising the step of recommending anticoagulation therapy or of recommending an intensification of anticoagulation therapy if the subject has been identified to be at risk to suffer from stroke.

4. The method according to claim 1, wherein the subject is human.

5. The method of according to claim 1, wherein the amounts of CES-2 and one or more biomarkers selected from the group consisting of a natriuretic peptide, ESM-1, ANG-2, and IGFBP7 are determined in step a), and wherein the method comprises the further steps of c) calculating a ratio of the amount of one or more biomarkers selected from the group consisting of a natriuretic peptide, ESM-1, ANG-2, and IGFBP7 as determined in step a) to the amount of CES-2 as determined in step a), and comparing said calculated ratio to a reference ratio.

6. A method for predicting the risk of stroke in a subject, comprising the steps of
a) determining the amount of CES-2 and/or the amount of one or more biomarkers selected from the group consisting of a natriuretic peptide, ESM-1, ANG-2, and IGFBP7 in a sample from the subject having a known clinical stroke risk score, and
b) assessing the clinical stroke risk score for said subject, and
c) predicting the risk of stroke based on the results of steps a) and b).

7. A method for improving the prediction accuracy of a clinical stroke risk score for a subject, comprising the steps of
a) determining the amount of CES-2 and/or the amount of one or more biomarkers selected from the group consisting of a natriuretic peptide, ESM-1, ANG-2, and IGFBP7, and
b) combining a value for the amount of CES-2 and/or the amount of one or more biomarkers selected from the group consisting of a natriuretic peptide, ESM-1, ANG-2, and IGFBP7 with the clinical stroke risk score, whereby the prediction accuracy of said clinical stroke risk score is improved.

8. A method for assessing the efficacy of an anticoagulation therapy of a subject, comprising the steps of
a) determining the amount of CES-2 and optionally one or more biomarkers selected from the group consisting of a natriuretic peptide, ESM-1, ANG-2, and IGFBP7 in a sample from the subject, and
b) comparing the amount of CES-2 and optionally one or more biomarkers selected from the group consisting of a natriuretic peptide, ESM-1, ANG-2, and IGFBP7 to a reference amount (or to reference amounts), whereby the risk of stroke is to be assessed.

9. A method according to claim 8, wherein a decreased amount of CES-2 is significant that anticoagulation therapy is not efficient, and wherein a normal or an increased amount of CES-2 is significant that anticoagulation therapy is effective.

10. A method for identifying a subject being eligible to the administration of at least one Fifanticoagulation medicament or being eligible for increasing the dosage of at least one anticoagulation medicament, comprising
a) determining the amount CES-2 and optionally one or more biomarkers selected from the group consisting of a natriuretic peptide, ESM-1, ANG-2, and IGFBP7 and
b) comparing the amount as determined in step a) with a reference amount,
whereby a subject being eligible to the administration of said at least one medicament or to an increased dosage of said at least one medicament is identified.

11. A method for monitoring a subject receiving an anticoagulation therapy, comprising the steps of
a) determining the amount of CES-2 and optionally one or more biomarkers selected from the group consisting of a natriuretic peptide, ESM-1, ANG-2, and IGFBP7 in a sample from the subject, and
b) comparing the amount of CES-2 and optionally of one or more biomarkers selected from the group consisting of a natriuretic peptide, ESM-1, ANG-2, and IGFBP7 to a reference amount (or to reference amounts), whereby the risk of stroke is to be assessed.

12. (canceled)

13. (canceled)

14. (canceled)

15. A kit comprising an agent which specifically binds to CES-2 and an agent which specifically binds to one or more biomarkers selected from the group consisting of a natriuretic peptide, ESM-1, ANG-2, and IGFBP7.

16. The method of according to claim 1, wherein the sample is selected from the group consisting of blood, serum and plasma.
